(19) United States
(12) Patent Application Publication
(10) Pub. No.: US 2008/0275318 A1
(43) Pub. Date: Nov. 6, 2008

(54) BIOSENSORS FOR MEASURING ANALYTES IN THE INTERSTITIAL FLUID

(75) Inventors: Alexander LASTOVICH, Raleigh, NC (US); Jacob Hartsell, Raleigh, NC (US); Javier Alarcon, Durham, NC (US); Glenn Vonk, Fuquay-Varina, NC (US); Bruce Clyde Roberts, Hillsborough, NC (US)

Correspondence Address: David W. Highet, VP & Chief IP Counsel;Becton, Dickinson and Company, (Morgan, Lewis & Bockius, LLP), 1 Becton Drive, MC 110, Franklin Lakes, NJ, 07417-1880, US

(73) Assignee: Becton, Dickinson and Company, Franklin Lakes, NJ (US)

(21) Appl. No.: 12/107,033

(22) Filed: Apr. 21, 2008

Related U.S. Application Data
(60) Provisional application No. 60/913,258, filed on Apr. 20, 2007.

(51) Int. Cl. A61B 5/1459; A61B 5/145

(52) U.S. Cl. 600/316; 600/365

(57) ABSTRACT

The invention relates to methods and devices for measuring blood glucose levels in a subject, where the methods and devices are designed for exposing a sensing mechanism to interstitial fluid in the subject.

### CROSS-REFERENCE TO RELATED APPLICATIONS

The application claims priority to U.S. Provisional Application No. 60/913,258, which was filed 20 Apr. 2007 and is incorporated by reference.

### BACKGROUND OF THE INVENTION

1. Field of the Invention

The invention relates to methods and devices for measuring blood glucose levels in a subject, where the methods and devices are designed for exposing a sensing mechanism to interstitial fluid in the subject.

2. Background of the Invention

A rapidly advancing area of biosensor development is the use of periplasmic binding proteins (PBPs), to accurately determine analyte, e.g., glucose, concentrations in biological samples. In particular, glucose-galactose binding proteins (GGBPs) are being employed as biosensors to measure analyte quantities in industrial and pharmacological physiological settings. PBPs are considered to be “reagentless” and can be used in a variety of settings including measuring glucose in monitoring diabetes, measuring amino acids in other metabolic diseases, such as histidase deficiency, as well as measuring arabinose during ethanol production from corn. Wild-type GGBPs, however, may not be the most ideal candidates for measuring or determining analyte concentrations for a variety of reasons. Biosensors comprising GGBPs would preferably be physically stable under conditions of use to generate a quantifiable signal upon glucose binding. When the intended use is as an implant to monitor in vivo glucose concentrations in diabetics, the proteins should preferably be stable and responsive at physiological temperatures.

An implantable biosensor could be used to constantly monitor the physiological state of a subject with a medical condition such as diabetes. The ideal biosensor for monitoring the levels of a ligand or target analyte, such as glucose, would need to be biocompatible so that the biosensor would not provoke an immune response or be subject to bio-fouling. To develop biosensors using analyte binding molecules, especially binding proteins, the binding molecules must be physically or chemically immobilized within a biosensor hydrogel in a manner that allows analyte-induced conformational change of the binding molecules. In addition, methods of chemical attachment are needed that prevent loss of the binding molecule, and provide a stable, continuous and reversible biosensor response to changing concentrations of the analyte of interest. The hydrogel matrix must be permeable to the analyte, prevent interference from other biomolecules, and be biocompatible and biostable.

Traditionally, implantable biosensors utilize large gauge sensors that cannot access shallow skin. This position within the skin for large gauge biosensors often results in a lengthy time lag when compared to glucose values from a capillary stick. This time-lag phenomenon is associated with many commercially available sensors. Current GGBP based continuous glucose monitoring sensors, which can access the subcutaneous space, may also display this lag. While the magnitude of the time lag may vary, the time lag is generally believed to be typically 10-20 minutes. Some systems withdraw interstitial fluid from the shallow skin to try and minimize the effect of the lag, but, to date, no biosensors have been implanted in the shallow skin space.

Since lag corrections usually introduce noise, an increase in applied rate constant will generally increase the noise observed. The true lag of a larger individual in vivo sensor appears not to be predictable and must be measured after the sensor is in place. This unpredictability is believed to be due to the heterogeneity of the subcutaneous space, i.e., how close to a vessel does the sensor tip end up in each test. In fact, to date, the FDA has not approved any continuous glucose monitoring system for therapy adjustment due in part to lack of acceptable correlation with capillary blood glucose tests.

### SUMMARY OF THE INVENTION

The present invention relates to biosensors for detecting blood glucose levels in a subject. The biosensors comprise a sensing mechanism that produces an optical signal in the presence of glucose, and the sensing mechanism preferably comprises a fluorescently labeled glucose-galactose binding protein (GGBP) that is preferably entrapped within a hydrogel matrix. The biosensor preferably comprises a needle, which comprises a body with a proximal end and a distal end, wherein the proximal end comprises a tip and wherein the needle tip houses the sensing mechanism. The needles used in the biosensors of the present invention are preferably 31 gauge needle or smaller. The body of the needle preferably also houses at least a portion of an optical conduit, wherein the optical conduit is in optical connectivity with the sensing mechanism.

The present invention also relates to methods for determining the blood levels of glucose in a subject. The methods comprise inserting to a depth of up to and including about 2 mm in a subject, a sensor that produces a detectable signal in the presence of glucose in the interstitial fluid of the subject of a subject, wherein the sensor produces a detectable signal in the presence of glucose present in the interstitial fluid of the subject. The sensor is maintained in the subject after insertion to allow exposure of at least a portion of the sensor to the interstitial fluid normally present in the intradermal space of the subject. The detectable signal is then measured and the signal produced by the sensor is correlated to the levels of blood glucose, thereby determining the levels of blood glucose in the subject.

### BRIEF DESCRIPTION OF THE DRAWINGS

FIG. 1 depicts in vivo high-rate glucose excursion with ultra-shallow 31Ga sensors, subcutaneous (subQ) 21Ga and 31Ga sensors using an R₀ update and a QR update compared to the core blood glucose and the ISF measured glucose.

FIG. 2 depicts the in vivo moderate glucose excursion comparing ISF to core blood glucose and capillary blood glucose.

FIG. 3 depicts in vivo moderate glucose excursion with ultra-shallow 31Ga sensors, subcutaneous (subQ) 21Ga and 31Ga sensors using an R₀ update and a QR update compared to the core blood glucose and capillary blood glucose.

FIG. 4 depicts in viva moderate glucose excursion with ultra-shallow 31Ga sensors compared to subcutaneous (subQ) 31Ga sensors using an R₀ update and a QR update against the core blood glucose and capillary blood glucose.

FIG. 5 depicts glucose tracking of several biosensors placed in the subcutaneous space of a pig under general anesthesia. The deeply implanted sensors fail to accurately track glucose after about 4 hours, post-implantation.

FIG. 6 depicts glucose tracking of several biosensors placed in the intradermal layer of a pig under general anesthesia. The shallow-depth implanted sensors accurately track glucose for at least 12 hours, post-implantation.

### DETAILED DESCRIPTION OF THE INVENTION

The present invention relates to biosensors for detecting blood glucose levels in a subject. The biosensors comprise a sensing mechanism that produces an optical signal in the presence of glucose, with the sensing mechanism preferably comprising a fluorescently labeled glucose-galactose binding protein (GGBP) that is preferably entrapped within a hydrogel matrix. The biosensor preferably comprises a needle, which comprises a body with a proximal end and a distal end, wherein the proximal end comprises a tip and wherein the needle tip houses the sensing mechanism. The needles used in the biosensors of the present invention are preferably 31 gauge needle or smaller. The body of the needle also preferably houses at least a portion of an optical conduit, wherein the optical conduit is in optical connectivity with the sensing mechanism.

The biosensors of the present invention comprise a sensing mechanism. The sensing mechanism used in the present invention preferably comprises a fluorescently labeled glucose-galactose binding protein (GGBP) that is entrapped within a hydrogel matrix. GGBP is a member of the well-known class of periplasmic binding proteins (PBPs), where these proteins are characterized by their three-dimensional configuration (tertiary structure), rather than the amino acid sequence (primary structure) of the protein. Each member of the class possesses a characteristic lobe-hinge-lobe motif. See Dwyer, M. A. and Helling a, H. W., Curr. Opin. Struct. Biol., 14:495-504 (2004), which is hereby incorporated by reference. The PBPs will normally bind an analyte specifically in a cleft region between the lobes of the PBP. Furthermore, the binding of an analyte in the cleft region will then cause a conformational change to the PBP that makes detection of the analyte possible. In general, the conformational changes to the PBP upon specific analyte binding are characterized by the two lobe regions to bend towards each other around and through the hinge region. See Quiocho, F. A. and Ledvina, P. S., Mol. Microbiol. 20:17-25 (1996), which is incorporated by reference. Examples of PBPs include, but are not limited to, glucose-galactose binding protein (GGBP), maltose binding protein (MBP), ribose binding protein (RBP), arabinose binding protein (ABP), dipeptide binding protein (DPBP), glutamate binding protein (GluBP), iron binding protein (FeBP), histidine binding protein (HBP), phosphate binding protein (PhosBP), glutamine binding protein (QBP), leucine binding protein (LBP), leucine-isoleucine-valine-binding protein (LIVBP), oligopeptide binding protein (OppA), or derivatives thereof, as well as other proteins that belong to the families of proteins known as periplasmic binding protein like I (PBP-like I) and periplasmic binding protein like II (PBP-like II).

In particular, the methods and devices of the present invention may comprise modified GGBPs. A “modified” protein, for example GGBP, is used to mean a protein that can be created by addition, deletion or substitution of one or more amino acids in the primary structure (amino acid sequence) of a reference protein or polypeptide. The terms “protein” and “polypeptide” are used interchangeably herein. The reference protein need not be a wild-type protein, but can be any protein that is targeted for modification for the purposes of, for example increasing thermal stability. Thus, the reference protein may be a protein whose sequence was previously modified over a wild-type protein. Of course, the reference protein may or may not be the wild-type protein from a particular organism. Furthermore, the term “wild-type protein” includes the wild-type protein with or without a “leader sequence.” Examples of GGBPs that can be used in the devices and methods of the present invention include but are not limited to those GGBPs described in U.S. application Ser. No. 11/738,442 (Pre-Grant Publication No. 2008/0044856), which is incorporated by reference. One particular example of a modified GGBP that may be useful in the present invention is described in Pre-Grant Publication No. 2008/0044856 is a GGBP termed “SM4” which is described in Example 4, at paragraph 125 and table 1. Other examples of GGBPs for use in the present invention include, but are not limited to, those that are described in U.S. Pat. No. 6,855,556, U.S. patent application Ser. No. 10/776,643 (Pre-Grant Publication No. 2005/0014290) all of which are incorporated by reference. One particular example of a modified GGBP that may be useful in the present invention is described in Pre-Grant Publication No. 2004/0118681 is a GGBP termed “W183C” which is the E. coli wild type GGBP with a single W183C mutation.

The GGBPs used in the biosensors of the present invention may also be modified, either by natural processes, such as post-translational processing, or by chemical modification techniques, which are well known in the art. Such modifications are well described in basic texts and in more detailed monographs, as well as in voluminous research literature. Modifications can occur anywhere in the polypeptide chain, including the peptide backbone, the amino acid side-chains and the amino or carboxyl termini. It will be appreciated that the same type of modification may be present in the same or varying degrees at several sites in a given polypeptide or protein. Also, a given polypeptide or protein may contain more than one modification. Examples of modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Polypeptides or proteins may even be branched as a result of ubiquitination, and they may be cyclic, with or without branching. (See, e.g., T. E. Creighton, Proteins-Structure And Molecular Properties, 2nd Ed., W. H. Freeman and Company, New York (1993); Wold, F., “Posttranslational Protein Modifications: Perspectives and Prospects”, in Posttranslational Covalent Modification Of Proteins, B. C. Johnson, Ed., Academic Press, New York (1983); Seifier et al., Methods in Enzymol, 182:626-646 (1990) and Rattan et al, Ann NY Acad, Sci., 663:48-62 (1992), all of which are incorporated herein by reference.

The GGBPs used in the biosensors of the present invention may be labeled with a labeling moiety. A “labeling moiety” as used herein, is intended to mean a chemical compound or ion that possesses or comes to possess a detectable signal. The labels used in the present invention may be used to indicate a conformational change in the lobe regions of the PBPs. Examples of changes in lobe regions include, but are not limited to, three-dimensional conformational changes changes in orientation of the amino acid side chains of proteinaceous binding domains, and redox states of the binding domains. With the addition/substitution of one or more residues into the primary structure of a protein, some of the labeling moieties used in the current methods and compositions can be attached through chemical means, such as reduction, oxidation, conjugation, and condensation reactions. Examples of residues commonly used to label proteins include, but are limited to, lysine and cysteine. For example, any thiol-reactive group can be used to attach labeling moieties, e.g., a fluorophore, to a naturally occurring or engineered cysteine in the primary structure of the polypeptide. U.S. Pat. No. 6,855,556, which is incorporated by reference, describes various cysteine mutations of PBPs. Also, for example, lysine residues can be labeled using succinimide ester derivatives of fluorophores. See Richieri, G. V. et al., J. Biol. Chem., 267: 23495-501 (1992) which is hereby incorporated by reference.

In specific embodiments, the labeling moiety can emit an optical signal. Numerous labels are known by those of skill in the art and include, but are not limited to, particles, fluorophores, haptens, enzymes and their colorimetric, fluorogenic and chemiluminescent substrates and other labels that are described in RICHARD P, HAUGLAND, MOLECULAR PROBES HANDBOOK OF FLUORESCENT PROBES AND RESEARCH PRODUCTS (9^{th} edition, CD-ROM, (September 2002), which is herein incorporated by reference.

A fluorophore of the present invention is any chemical moiety that exhibits an absorption maximum at or beyond 280 nm, and when covalently attached to a protein or other reagent retains its spectral properties. Fluorophores of the present invention include, without limitation; a pyrene (including any of the corresponding derivative compounds disclosed in U.S. Pat. No. 5,132,432, incorporated by reference), an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a cyanine, a carbocyanine (including any corresponding compounds in U.S. Pat. Nos. 4,981,977; 5,268,486; 5,569,587; 5,569,766; 5,486,616; 5,627,027; 5,808,044; 5,877,310; 6,002,003; 6,004,536; 6,008,373; 6,043,025; 6,127,134; 6,130,094; 6,133,445; 6,664,047; 6,974,873 and 6,977,305; and publications WO 02/26891, WO 97/40104, WO 99/51702, WO 01/21624; EP 1 065 250 A1, incorporated by reference), a carbostyryl, a porphyrin, a salicylate, an anthranilate, an azulene, a perylene, a pyridine, a quinoline, a borapolyazaindacene (including any corresponding compounds disclosed in U.S. Pat. No. 4,774,339; 5,187,288; 5,248,782; 5,274,113; and 5,433,896, incorporated by reference), a xanthene (including any corresponding compounds disclosed in U.S. Pat. Nos. 6,162,931; 6,130,101; 6,229,055; 6,339,392; 5,451,343 and 6,716,979, incorporated by reference), an oxazine (including any corresponding compounds disclosed in U.S. Pat. No. 4,714,763, incorporated by reference) or a benzoxazine, a carbazine (including any corresponding compounds disclosed in U.S. Pat. No. 4,810,636, incorporated by reference), a phenalenone, a coumarin (including an corresponding compounds disclosed in U.S. Pat. Nos. 5,696,157; 5,459,276; 5,501,980 and 5,830,912, incorporated by reference), a benzofuran (including an corresponding compounds disclosed in U.S. Pat. Nos. 4,603,209 and 4,849,362, incorporated by reference) and benzphenalenone (including any corresponding compounds disclosed in U.S. Pat. No. 4,812,409, incorporated by reference) and derivatives thereof. As used herein, oxazines include resorufins (including any corresponding compounds disclosed in U.S. Pat. No. 5,242,805, incorporated by reference), aminooxazinones, diaminooxazines, and their benzo-substituted analogs. Additional labeling moieties include, but are not limited to, those compounds that are described in United States Patent Publication No. 2006/0280652, published 14 Dec. 2006 and PCT Publication No. WO 2006/025887, which are incorporated by reference.

When the fluorophore is a xanthene, the fluorophore is optionally a fluorescein, a rhodol (including any corresponding compounds disclosed in U.S. Pat. Nos. 5,227,487 and 5,442,045, incorporated by reference), or a rhodamine (including any corresponding compounds in U.S. Pat. Nos. 5,798,276; 5,846.737 and 6,562,632, incorporated by reference). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171, incorporated by reference). Alternatively, the fluorophore is a xanthene that is bound via a linkage that is a single covalent bond at the 9-position of the xanthene. Preferred xanthenes include derivatives of 3H-xanthen-6-ol-3-one attached at the 9-position, derivatives of 6-amino-3H-xanthen-3-one attached at the 9-position, or derivatives of 6-amino-3H-xanthen-3-imine attached at the 9-position.

Fluorophores for use in the present invention include, but are not limited to, xanthene (rhodol, rhodamine, fluorescein and derivatives thereof) coumarin, cyanine, pyrene, oxazine and borapolyazaindacene. Most preferred are sulfonated xanthenes, fluorinated xanthenes, sulfonated coumarins, fluorinated coumarins and sulfonated cyanines. The choice of the fluorophore will determine the absorption and fluorescence emission properties of the GGBP or other labeling reagent complex. Physical properties of a fluorophore label include spectral characteristics (absorption, emission and stokes shift), fluorescence intensity, lifetime, polarization and photo-bleaching rate all of which can be used to distinguish one fluorophore from another.

Typically the fluorophore contains one or more aromatic or heteroaromatic rings, that are optionally substituted one or more times by a variety of substituents, including without limitation, halogen, nitro, cyano, alkyl, perfluoroalkyl, alkoxy, alkenyl, alkynyl, cycloalkyl, arylalkyl, acyl, aryl or heteroaryl ring system, benzo, or other substituents typically present on fluorophores known in the art.

Specific examples of fluorophore labels are selected from the group consisting of fluorescein, coumarins, rhodamines, 5-TMRIA (tetramethylrhodamine-5-iodoacetamide), (9-(2(or 4)-(N-(2-maleimdylethyl)-sulfonamidyl)-4(or 2)-sulfophenyl)-2,3,6,7,12,13,16,17-octahydro-(1-H,5H,11H,15H-xantheno(2,3,4-ij:5,6,7-i′j′)diquinolizin-18-ium salt) (Texas Red®), 2-(5-(1-(6-(N-(2-maleimdylethyl)-amino)-6-oxohexyl)-1,3-dihydro-3,3-dimethyl-5-sulfo-2H-indol-2-ylidene)-1,3-propyldienyl)-1-ethyl-3,3-dimethyl-5-sulfo-3H-indolium salt (Cy™3), N,N′-dimethyl-N-(iodoacetyl)-N′-(7-nitrobenz-2-oxa-1,3-diazol-4-yl)ethylenediamine (IANBD amide), N-((2-(iodoacetoxy)ethyl)-N-methyl)amino-7-nitrobenz-2-oxa-1,3-diazole (IANBD ester), 6-acryloyl-2-dimethylaminonaphthalene (acrylodan), pyrene, 6-amino-2,3-dihydro-2-(2-((iodoacetyl)amino)ethyl)-1,3-dioxo-1H-benz(de)isoquinoline-5,8-disulfonic acid salt (lucifer yellow), 2-(5-(1-(6-(N-(2-maleimdylethyl)-amino)-6-oxohexyl)-1,3-dihydro-3,3-dimethyl-5-sulfo-2H-indol-2-ylidene)-1,3-pentadienyl)-1-ethyl-3,3-dimethyl-5-sulfo-3H-indolium salt (Cy™5), 4-(5-(4-dimethylaminophenyl)oxazol-2-yl)phenyl-N-(2-bromoacetamidoethyl)sulfonamide (Dapoxyl®(2-bromoacetamidoethyl)sulfonamide)), (N-(4,4-difluoro-1,3,5,7-tetramethyl-4-bora-3a,4a-diaza-s-indacene-2-yl)iodoacetamide (BODIPY® 507/545 IA), N-(4,4-difluoro-5,7-diphenyl-4-bora-3a,4a-diaza-s-indacene-3-propionyl)-N′-iodoacetylethylenediamine (BODIPY 530/550 IA), 5-((((2-iodoacetyl)amino)ethyl) amino)napthalene-1-sulfonic acid (1,5-IAEDANS), and carboxy-X-rhodamine, 5/6-iodoacetamide (XRIA 5,6). Another example of a label is BODIPY-FL-hydrazide. Other luminescent labels include lanthanides such as europium (Eu3+) and terbium (Tb3+), as well as metal-ligand complexes of ruthenium [Ru(II)], rhenium [Re(I)], or osmium [Os(II)], typically in complexes with diimine ligands such as phenanthroline. United States Patent Publication No. 2006/0280652, published 14 Dec. 2006, which is incorporated by reference, discloses additional fluorophores that may be useful for the present invention.

Fluorescent proteins also find use as labels for the labeling reagents of the present invention. Thus, in one specific embodiment, the reference PBP, such as a GGBP, is a fusion protein comprising a functional GGBP and a fluorescent protein, where the fluorescent protein acts as at least one label. The PBPs would, in turn, comprise a fluorescent protein. In another embodiment, the proteins of the current invention may comprise two, three, four or more fluorescent proteins. If the fusion proteins of the current invention contain more than one fluorescent protein, the fluorescent proteins may or may not be chemically identical. Fluorescent proteins are easily recognized in the art. Examples of fluorescent proteins that are part of fusion proteins of the current invention include, but are not limited to, green fluorescent proteins (GFP, AcCEP, ZsGreen), red-shifted GFP (rs-GFP), red fluorescent proteins (RFP, including DsRed2, HcRed1, dsRed-Express), yellow fluorescent proteins (YFP, Zsyellow), cyan fluorescent proteins (CFP, AmCyan), a blue fluorescent protein (BFP) and the phycobiliproteins, as well as the enhanced versions and mutations of these proteins. For some fluorescent proteins enhancement indicates optimization of emission by increasing the proteins' brightness or by creating proteins that have faster chromophore maturation. These enhancements can be achieved through engineering mutations into the fluorescent proteins.

The fluorescent proteins, especially phycobiliprotein, are particularly useful for creating tandem dye labeled labeling reagents. In one embodiment of the current invention, therefore, the measurable signal of the fusion protein is actually a transfer of excitation energy (resonance energy transfer) from a donor molecule to an acceptor molecule. In particular, the resonance energy transfer is in the form of fluorescence resonance energy transfer (FRET). When the proteins used in the present invention utilize FRET to measure of quantify analyte(s), the fusion protein can be the donor or the acceptor. The terms “donor” and “acceptor,” when used in relation to FRET, are readily understood in the art. Namely, a donor is the molecule that will absorb a photon of light and subsequently initiate energy transfer to the acceptor molecule. The acceptor molecule is the molecule that receives the energy transfer initiated by the donor and, in turn, emits a photon of light. The efficiency of FRET is dependent upon the distance between the two fluorescent partners and can be expressed mathematically by: E=R₀⁶/(R₀⁶+r⁶), where E is the efficiency of energy transfer, r is the distance (in Angstroms) between the fluorescent donor/acceptor pair and R₀ is the Förster distance (in Angstroms). The Förster distance, which can be determined experimentally by readily available techniques in the art, is the distance at which FRET is half of the maximum possible FRET value for a given donor/acceptor pair. A particularly useful combination is the phycobiliproteins disclosed in U.S. Pat. Nos. 4,520,110; 4,859,582; 5,055,556, incorporated by reference, and the sulforhodamine fluorophores disclosed in U.S. Pat. No. 5,798,276, or the sulfonated cyanine fluorophores disclosed in U.S. Pat. Nos. 6,977,305 and 6,974,873; or the sulfonated xanthene derivatives disclosed in U.S. Pat. No. 6,130,101, incorporated by reference and those combinations disclosed in U.S. Pat. No. 4,542,104, incorporated by reference.

As stated previously, the fluorescently labeled GGBPs are entrapped within a hydrogel matrix. As used herein, the term “entrap” and variations thereof is used interchangeably with “encapsulate” and is used to mean that the binding molecule is immobilized within or on the constituents of the matrix. As used herein, “matrix” refers to essentially a three-dimensional environment which has at least one PBP immobilized therein for the purpose of measuring a detectable signal from ligand-protein interaction. Examples of matrices that are capable of entrapping the GGBPs are disclosed in United States Patent Publication No. 2005/0923155, published 27 Oct. 2005, which is incorporated by reference. The relationship between the constituents of the matrix and the PBPs include, but are not limited to, covalent, ionic, and Van der Wals interactions and combinations thereof. The spatial relationship between the matrix and the PBPs includes heterogeneous and homogeneous distribution within and or upon any or all of the matrix volume. The hydrogel matrix may be comprised of organic, inorganic, glass, metal, plastic, or combinations thereof.

The hydrogel matrix can be in any desirable form or shape including one or more of disk, cylinder, patch, nanoparticle, microsphere, porous polymer, open cell foam, and combinations thereof providing it permits permeability to analyze. The hydrogel matrix additionally prevents leaching of the protein from the sensing mechanism. The hydrogel matrix permits light from optical sources or any other interrogating light to or from the reporter group to pass through the biosensor. When used in an in vivo application, the biosensor will be exposed to a substantially physiological range of analyte and determination or detection of a change in analyte concentration would be desired whereas the determination or detection includes continuous, programmed, and episodic detection means.

The hydrogel matrix may be prepared from biocompatible materials or incorporates materials capable of minimizing adverse reactions with the body. Adverse reactions for implants include inflammation, protein fouling, tissue necrosis, immune response and leaching of toxic materials. Such materials or treatments are well known and practiced in the art, for example as taught by Quinn, C. P.; Pathak, C. P.; Heller, A.; Hubbell, J. A. Biomaterials 1995, 16(5), 389-396, and Quinn, C. A. P.; Connor, R. E.; Heller, A. Biomaterials 1997, 18(24), 1665-1670.

As used herein, the term “hydrogel” is used to indicate a water-insoluble, water-containing material. Numerous hydrogels may be used in the present invention. The hydrogels may be, for example, polysaccharides such as agarose, dextran, carrageenan, alginic acid, starch, cellulose, or derivatives of these such as, e.g., carboxymethyl derivatives, or a water-swellable organic polymer such as, e.g., polyvinyl alcohol, polyacrylic acid, polyacrylamide, polyethylene glycol, copolymers of styrene and maleic anhydride, copolymers of vinyl ether and maleic anhydride and derivates thereof. Derivatives providing for covalently crosslinked networks are preferred. Synthesis and biomedical and pharmaceutical applications of hydrogels have been described by a number of researchers. (See, e.g. “Biosensors Fundamentals and Applications”, edited by A. D. F. Turner, I. Karube and G. S. Wilson; published from Oxford University Press, in 1988). An exemplary hydrogel matrix derived from a water-soluble, UV crosslinkable polymer comprises poly(vinyl alcohol),N-methyl-4(4′-formylstyryl)pyridinium methosulphate acetal (CAS Reg. No. [107845-59-0]) available from PolyScience Warrington, Pa.

The polymers that are to be used in the hydrogel matrices used in the present invention may be functionalized. Of course, polymers used in other matrices may also be functionalized. That is, the polymers or monomers comprising the polymers should possess reactive groups such that the hydrogel matrices are amenable to chemical reactions, e.g., covalent attachment. As used herein and throughout, a “reactive group” is a chemical group that can chemically react with a second group. The reactive group of the polymer or monomers comprising the polymer may itself be an entire chemical entity or it may be a portion of an entire chemical entity, including, but not limited to, single atoms or ions. Further, the second group with which the reactive group is capable of reacting can be the same or different from the reactive group of the polymer or monomers comprising the polymers. Examples of reactive groups include, but are not limited to, halogens, amines, amides, aldehydes, acrylates, vinyls, hydroxyls and carboxyls. In one embodiment, the polymers or monomers comprising the polymers of the hydrogel should be functionalized with carboxylic acid, sulfate, hydroxy or amine groups. In another embodiment of the present invention, the polymers or monomers comprising the polymers of the hydrogel are functionalized with one or more acrylate groups. In one particular embodiment, the acrylate functional groups are terminal groups. The reactive groups of the polymers or monomers comprising the polymers of the matrix may be reactive with any component of the matrix portion of the biosensor, such as, but not limited to, another polymer or monomer within the matrix, a binding protein and an additive.

Suitable polymers which may be used in the present invention include, but are not limited to, one or more of the polymers selected from the group consisting of poly(vinyl alcohol), polyacrylamide, poly (N-vinyl pyrolidone), poly(ethylene oxide) (PEO), hydrolysed polyacrylonitrile, polyacrylic acid, polymethacrylic acid, poly(hydroxyethyl methacrylate), polyurethane polyethylene amine, poly(ethylene glycol) (PEG), cellulose, cellulose acetate, carboxy methyl cellulose, alginic acid, pectinic acid, hyaluronic acid, heparin, heparin sulfate, chitosan, carboxymethyl chitosan, chitin, collagen, pullulan, gellan, xanthan, carboxymethyl dextran, chondroitin sulfate, cationic guar, cationic starch as well as salts and esters thereof. The polymers of the hydrogel matrix may also comprise polymers of two or more distinct monomers. Monomers used to create copolymers for use in the matrices include, but are not limited to acrylate, methacrylate, methyl methacrylate, methacrylic acid, alkylacrylates, phenylacrylate, hydroxyalkylacrylates, hydroxyalkylmethacrylates, aminoalkylacrylates, aminoalkylmethacrylates, alkyl quaternary salts of aminoalkylacrylamides, alkyl quaternary salts of aminoalkylmethacrylamides, and combinations thereof. Polymer components of the matrix may, of course, include blends of other polymers.

In one embodiment, the hydrogel is comprised of poly(ethylene glycol) dimethacrylate (PEGDMA). PEGDMA is commercially available in a variety of molecular weights. For example. PEGDMA is available from at least Aldrich Chemical Co. (Milwaukee, Wis. USA) and from Polysciences, Inc. (Warrington, Pa., USA) and can be synthesized in an assortment of molecular weights. In one embodiment, the molecular weight of PEGDMA used in the hydrogels of the present invention is from about 400 to about 4000. In a more specific embodiment, the molecular weight of the PEGDMA in the hydrogels is about 1000.

In another embodiment, the hydrogels comprise PEGDMA and at least one acrylate. As used herein, the term acrylate is well understood in the art. Specifically, acrylates are compounds, including but not limited to polymers, comprising the acrylic group (HC₂═CH—C(═O). Examples of acrylates include, but are not limited to, acrylic acid, ethyl acrylate, methacrylic acid, methyl methacrylic acid and acrylamides. In another specific embodiment, the hydrogels comprise more than one acrylate. In a more specific embodiment, the hydrogels comprise a mixture of methacrylate and methyl methacrylate. Examples of hydrogels that may be used in the present invention include those compositions described in the U.S. Non-Provisional Application claiming priority to Application No. 60/913,261, the filing date of which was 21 Apr. 2008 and the priority date of which was 20 Apr. 2008, entitled “HYDROGEL COMPOSITIONS” (Attorney Docket No. P-7202), which is incorporated by reference.

The polymers used in the hydrogel matrices can be modified to contain nucleophilic or electrophilic groups. Indeed, the polymers used in the present invention may further comprise polyfunctional small molecules that do not contain repeating monomer units but are polyfunctional, i.e., containing two or more nucleophilic or electrophilic functional groups. These polyfunctional groups may readily be incorporated into conventional polymers by multiple covalent bond-forming reactions. For example, PEG can be modified to contain one or more amino groups to provide a nucleophilic group. Examples of other polymers that contain one or more nucleophilic groups include, but are not limited to, polyamines such as ethylenediamine, tetramethylenediamine, pentamethylenediamine, hexamethylenediamine, bis-(2-hydroxyethyl) amine, bis-(2-aminoethyl)amine, and tris-(2-aminoethyl)amine. Examples of electrophilic groups include but are not limited to, succinimide esters, epoxides, hydroxybenzotriazole esters oxycarbonylimidazoles, nitrophenyl carbonates, tresylates, mesylates, tosylates, carboxylates, and isocyanates. In one embodiment, the composition comprises a bis-amine-terminated poly(ethylene glycol).

The polymers should be capable of crosslinking, either physically or chemically, to form a hydrogel. Physical crosslinking includes, but is not limited to, such non-chemical processes as radiation treatment such as electron beams, gamma rays, x-rays, ultraviolet light, anionic and cationic treatments. The crosslinking of the polymers may also comprise chemical crosslinking, such as covalent crosslinking. For example, a chemical crosslinking system may include, but is not limited to, the use of enzymes, which is well-known in the art. Another example of the chemical covalent crosslinking comprises the use of peroxide. Chemical crosslinking may occur when a crosslinking reagent reacts with at least two portions of a polymer to create a three-dimensional network. Covalent crosslinking may also occur when multifunctional monomers are used during the crosslinking process. For example, an acrylate monomer may be polymerized with a bifunctional acrylate monomer to form a crosslinked polymer. Any crosslinking reagent will be suitable for the present invention, provided the crosslinking reagent will at least partially dissolve in water or an organic solvent and can form the crosslinked polymer. For example, if the polymer is an amine-terminated PEG, the crosslinking reagent should be capable of reacting with the PEG-amine groups and be substantially soluble in water. In another example, (hydroxyethyl methacrylate) and methacrylic acid monomers can be polymerized with poly(ethylene glycol)-bis-alkylacrylate crosslinking agent in water or in dimethylformide to form polymeric hydrogels.

If the polymers to be crosslinked are functionalized with nucleophilic groups, such as amines (primary, secondary and tertiary), thiols, thioethers, esters, nitrites, and the like, the crosslinking reagent can be a molecule containing an electrophilic group. Examples of electrophilic groups have been described herein. Likewise, if polymers to be crosslinked are functionalized with electrophilic groups, the crosslinking reagent can be a molecule containing a nucleophilic group. It is understood that one skilled in the art can exchange the nucleophilic and electrophilic functional groups as described above without deviating from the scope of the present embodiment. It is also understood that the binding molecule can provide the requisite nucleophilic and electrophilic functional groups. For example, where the binding molecule is a protein, the nucleophilic and electrophilic functional groups may be present as naturally occurring amino acids in the protein, or may be introduced to the protein using chemical techniques described herein. Other general methods for preparing or crosslinking polymers to form hydrogel matrices are well known in the art. For example, Ghandehari H., et al., J. Macromol. Chem. Phys. 197: 965 (1996); and Ishihara K, et al., Polymer J., 16: 625 (1984), all of which are hereby incorporated by reference, report the formation of hydrogels. Hydrogel matrix can be applied to each sensor tip, e.g. a needle, and cured under a Hg lamp, with wavelength of >360 nm, for about 15 seconds.

In one embodiment of the present invention, the binding molecules are covalently attached to, i.e., entrapped within a hydrogel. The covalent attachment of the binding molecule to the hydrogel should not interfere with the binding of the binding molecule to the target ligand. Furthermore, the covalent attachment of the binding molecule to the hydrogel should be resistant to degradation. The functional group in one embodiment, a polymer or other component of the hydrogel serves to couple the binding molecule to the hydrogel. The coupling of the binding molecule to the hydrogel can be accomplished in any number of ways. For example, coupling reactions between the hydrogel and binding molecule include, but are not limited to, diazonium coupling, isothiocyano coupling, hydrazide coupling, amide formation, disulfide coupling, maleic anhydride coupling, thiolactone coupling, and dichlotriazine coupling. These coupling reactions between two functional groups are well documented, and are considered well known to those skilled in the art. For example, an amino functional group in a binding molecule can be covalently coupled to a carboxyl functional group of one or more components of a hydrogel using coupling agents such as 1-ethyl-3-(3-dimethylaminopropyl) carbodiimide hydrochloride (EDC) or dicyclohexylcarbodiimide (DCC). It is understood that the amino and carboxyl functional groups of the binding molecule and one or more components of the hydrogel as described above can be transposed without deviating from the scope of the embodiment.

The biosensors of the present invention may also comprise a needle. The needle comprises a longitudinal body with a proximal and distal end. At the proximal end of the needle is a tip. Often times, the tip of the needle may be beveled. One example of a needle that can be used in the biosensors of the present invention is a 31 gauge needle. Needles smaller than 31 gauge may also be used in the present invention. As used herein a needle that is “smaller than a 31 gauge needle” is a needle with an outer diameter that is less than the outer diameter of a 31 gauge needle. As is well understood in the art, the diameter of a needle is correlated to its outside diameter using the industry-standard Birmingham Wire Gauge.

The biosensors of the present application can be placed into the intradermal layer of the skin of the subject, thereby exposing the sensing mechanism to the interstitial fluid of the subject. As used herein, the “intradermal layer” of the skin is used as it is in the art. Namely, the intradermal layer is portion of skin that includes the epidermal and the dermal layers of the skin, but not the subcutaneous layer. Other sensors, such as subcutaneous implants, typically demonstrate a lag time in measuring the levels of blood glucose in a subject. Shallow depth skin tissue glucose levels appear to have little, if any, time lag when compared to the glucose value in blood. This shallow depth penetration enables the sampling of interstitial fluids for accurate glucose concentrations levels, with almost no lag time. In one embodiment, therefore, the needle of the biosensor is capable of penetrating a subject's skin at shallow depths. In particular, the needles can penetrate below the stratum corneum of the skin of a subject to a depth of about 2 mm or less from the surface of the skin. In a more specific embodiment, the needles can penetrate the skin of a subject to a depth of about 1 mm or less from the surface of the skin. In a more specific embodiment, the needles can penetrate the skin of a subject to a depth of about 0.8 mm or less from the surface of the skin. In particular, larger gauge sensors, e.g., 21 and 25 gauge needles are too large to be accurately placed in such shallow skin depths. In general, lag time is reduced as the needle is implanted at a more shallow depth. Thus, while the biosensors and methods of the present invention will work at any depth within the intradermal layer of the skin, a more shallow depth may be desirable if a short lag time is desired.

The body of the needle may also house at least a portion of an optical conduit. The optical conduit, which may vary in length from approximately 0.1 cm to 5 meters, should be in optical connectivity with the sensing element such that the conduit couples light into and out of an optical system and into and out of the sensing mechanism. For example, the optical conduit may be a lens, a reflective channel, a needle, or an optical fiber. The optical fiber may be either a single strand of optical fiber (single or multimode) or a bundle of more than one fiber. In one embodiment, the bundle of fibers is bifurcated. The fiber may be non-tapered or tapered.

An optical system may be connected to the distal end of the optical conduit. The optical system consists of a combination of one or more excitation sources and one or more detectors. It may also consist of filters, dichroic elements, a power supply, and electronics for signal detection and modulation. The optical system may optionally include a microprocessor.

The optical system interrogates the sample either continuously or intermittently by coupling one or more interrogating wavelengths of light into the optical conduit. The one or more interrogating wavelengths then pass through the optical conduit and illuminate the sensing mechanism. A change in glucose concentration may result in a change of the wavelength, intensity, lifetime, energy transfer efficiency, and/or polarization of the luminescence of the reporter group, which is a part of the sensing mechanism. The resulting changes in optical properties of the signaling moiety pass back through the optical conduit to the optical system where at least one of the optical properties is detected, interpreted, and stored and/or displayed. In certain embodiments, the optical system comprises multiple excitation sources. One or more of these sources may be modulated to permit dynamic signal processing of the detected signal, thereby enhancing signal-to-noise and detection sensitivity. Modulation may also be used to reduce power consumption by the device or to increase the lifetime of the sensing element by minimizing undesirable phenomena such as photobleaching. The optical system can also include one or more electromagnetic energy detectors that can be used for detecting the luminescence signal from the reporter and optional reference groups as well as for internal referencing and/or calibration. The overall power consumption of the optical system may be kept small to permit the device to be operated using battery power.

Once exposed to the interstitial fluids of the subject, the sensing mechanism can be interrogated with light through the optical conduit. The optical conduit then returns at least one optical signal from the fluorescent label, e.g., intensity, to the optical system. The optical signal is generated by the fluorescent label from the labeled GGBP when glucose is present in the interstitial fluid. This generated signal can be correlated to blood glucose levels with little, if any, lag time.

The interstitial fluid glucose levels in very shallow skin are closer to natural blood levels, and only very small sensors can measure the values without fluid removal or mechanical stimulation. In addition, the small sensors of the present invention in shallow skin have the ability to respond rapidly and to match blood glucose values with glucose rates of change >10 mg %/min. Blood glucose levels in healthy humans usually change at less than 1 mg %/min, but levels may change as rapidly as 4 mg %/min after a meal. Thus, the small sensors of the present invention do not suffer the problems associated with time lag from traditional blood glucose monitors, because the necessary time constants appear to be more consistent and show very short lags such that any error in a pre-determined value translates into smaller glucose error.

The generated signal is due to the presence of glucose in the interstitial fluid of the subject, and the interstitial levels of glucose are related to blood glucose levels in the subject. In general, the interstitial fluid levels are directly related to the blood glucose levels of the subject. Thus, in one embodiment of the present invention, the interstitial fluid glucose levels serve as a surrogate for the blood glucose levels of the subject where the measurement of the interstitial fluid glucose levels are taken as the blood glucose levels of subject. In another embodiment, the interstitial fluid glucose levels may be correlated to the blood glucose levels. For example, an algorithm may be applied to the measured interstitial fluid glucose levels to arrive at the blood glucose levels of the subject. The algorithm or other operation used to transform the measured interstitial fluid glucose levels may be applied to the concentration or levels of glucose. The algorithm may also be applied to adjust for time lag between interstitial fluid levels and blood glucose levels.

The biosensors of the present invention are useful in a variety of applications, such as industrial processes, and as components of biosensors to detect, monitor or measure analyte quantities, i.e., glucose, in a sample. As used herein, a sample can be any environment that may be suspected of containing the analyte to be measured. Thus, a sample includes, but is not limited to, a solution, a cell, a body fluid, a tissue or portion thereof, and an organ or portion thereof. Examples of biological fluids to be assayed include, but are not limited to, blood, urine, saliva, synovial fluid, interstitial fluid, cerebrospinal fluid, lymphatic fluids, bile and amniotic fluid. The scope of the methods of the present invention should not be limited by the type of body fluid assayed. The terms “subject” and “patient” are used interchangeably herein and are used to mean an animal, particularly a mammal, more particularly a human or nonhuman primate.

The samples may or may not have been removed from their native environment. Thus, the portion of sample assayed need not be separated or removed from the rest of the sample or from a subject that may contain the sample. For example, the blood or interstitial fluid of a subject may be assayed for glucose without removing any of the blood or interstitial from the patient. Of course, the sample may also be removed from its native environment. Furthermore, the sample may be processed prior to being assayed. For example, the sample may be diluted or concentrated; the sample may be purified and/or at least one compound, such as an internal standard, may be added to the sample. The sample may also be physically altered (e.g., centrifugation, affinity separation) or chemically altered (e.g., adding an acid, base or buffer, heating) prior to or in conjunction with the methods of the current invention. Processing also includes freezing and/or preserving the sample prior to assaying.

The biosensors of the present invention may be used in a continuous or episodic setting. As used herein, the term “continuous” as it relates to glucose monitoring or glucose sensing indicates that the same sensing mechanism can be used repeatedly at virtually any time interval to generate a measured blood glucose level. The biosensor need not actually produce a constant signal for the sensor to be considered “continuous,” provided that the sensing mechanism is capable of returning a measured glucose value at any time to the user, on demand. In contrast, an episodic sensor is not capable of repeated uses at virtually any time interval. One example of an episodic sensor includes, but is not limited to, glucose strips that are disposed after one use. In one embodiment of the present invention, the biosensors are implanted and used in a continuous manner, such that a user can, at any time, interrogate the sensing mechanism with light to produce a return signal, where the return signal is signal generated in response to interstitial fluid glucose levels.

The implanted biosensors are capable of providing continuous monitoring of glucose for the entire time they are implanted in the subject. In one embodiment, the present invention relates to the biosensors remaining implanted in the subject for at least or up to 2, 4, 6, 8, 10, 12, 14, 16, 18, 20 or 22 hours and the implanted biosensors provide continuous blood glucose monitoring. In one embodiment, the present invention relates to the biosensors remaining implanted in the subject for at least or up to one day (24 hours) and the implanted biosensors provide continuous blood glucose monitoring. In another embodiment, the biosensors remain implanted in the subject for at least or up to two days (48 hours) and the implanted biosensors provide continuous blood glucose monitoring. In another embodiment, the biosensors remain implanted in the subject for at least or up to three days (72 hours) and the implanted biosensors provide continuous blood glucose monitoring. In another embodiment, the biosensors remain implanted in the subject for at least or up to four days (96 hours) and the implanted biosensors provide continuous blood glucose monitoring. In another embodiment, the biosensors remain implanted in the subject for at least or up to five days (120 hours) and the implanted biosensors provide continuous blood glucose monitoring. In another embodiment, the biosensors remain implanted in the subject for at least or up to six days (144 hours) and the implanted biosensors provide continuous blood glucose monitoring. In another embodiment, the biosensors remain implanted in the subject for at least or up to seven days (168 hours) and the implanted biosensors provide continuous blood glucose monitoring. In another embodiment, the biosensors remain implanted in the subject for at least or up to eleven days (264 hours) and the implanted biosensors provide continuous blood glucose monitoring.

The shallow depth biosensors of the present invention have advantages over traditional sensors that are implanted into the subcutaneous space of the subject. Traditional glucose monitors are normally implanted in the subcutaneous space, where these sensors are dependent on peripheral blood flow. Because the sensors of the present invention measure glucose in the interstitial fluid and not blood, the small sensors of the present invention can determine blood glucose levels even when the subject's peripheral blood flow is compromised or altered in some way. Thus, in one embodiment, the biosensors are implanted into a subject, where the peripheral blood flow of the subject is decreased compared to the subject's normal peripheral blood flow. Examples of conditions or physiological stress situations in which a subject's peripheral blood flow may be decreased include, but are not limited to, general anesthesia, hypothermia, hypotension, coma, allergic reaction, and shock.

In that vein, the present invention also relates to methods for determining the blood levels of glucose in a subject. The methods comprise inserting to a depth of about 0.1 mm to about 2 mm in a subject, a sensor that produces a detectable signal in the presence of glucose in the interstitial fluid of the subject of a subject, wherein the sensor produces a detectable signal in the presence of glucose present in the interstitial fluid of the subject. In one embodiment, the sensor is inserted between about 0.1 mm and 2 mm below the surface the skin. In another embodiment, the sensor is inserted into the subject at a depth of between about 0.1 mm and 1 mm below the surface the skin. In another embodiment, the sensor is inserted into the subject at a depth of between about 0.1 mm and 0.8 mm below the surface the skin. The sensor is maintained in the subject after insertion to allow exposure of at least a portion of the sensor to the interstitial fluid normally present in the intradermal space of the subject. The detectable signal is then measured and the signal produced by the sensor is correlated to the levels of blood glucose, thereby determining the levels of blood glucose in the subject.

The methods can be performed in a continuous or episodic fashion, as described herein. Moreover, the methods can be performed for at least or up to 2, 4, 6, 8, 10, 12, 14, 16, 18, 20, 22, 24, 36, 48, 60, 72, 84, 96, 108, 120, 132, 144, 156 or 168 hours. The methods can also be performed in any setting, including but not limited to, a normal subject or a subject with decreased peripheral blood flow, such as when the subject is under general anesthesia or when the subject is under a physical stress as disclosed herein.

The following examples are illustrative and are not intended to limit the scope of the invention described herein.

### EXAMPLES

### Example 1

### Glucose Tracking for Shallow-Depth Sensors Implanted in Various Layers of Skin

Using a simplified algorithm with no time lag applied, using the small sensors of the present invention generally resulted in lower mean percent error (MPE) values. Table I shows the average MPE for a number of sensors using only one update (fingerstick) and the pre-calibration parameters no time adjustment. If a student's t-test is applied for statistical significance (significance is p<0.05 and using a 1 sided type 3 t-test) p=0.028 when comparing a 31 gauge biosensor implanted to a shallow depth (“Shallow 31”), versus a 31 gauge biosensor implanted to a deep (subcutaneous) depth (“Deep 31”). When comparing “Deep 31” to “Deep 21”(a 21 gauge needle implanted into the subcutaneous space), p=0.04, showing that differences in MPE between the two sensors are statistically significant.

TABLE I

| Shallow 31 | Deep 31 | Deep 21 |
|---|---|---|
| N = 14 | N = 12 | N = 12 |
| MPE (CV %) | MPE (CV %) | MPE (CV %) |
| 16.3 (17) | 25.4 (58) | 37.3 (46) |

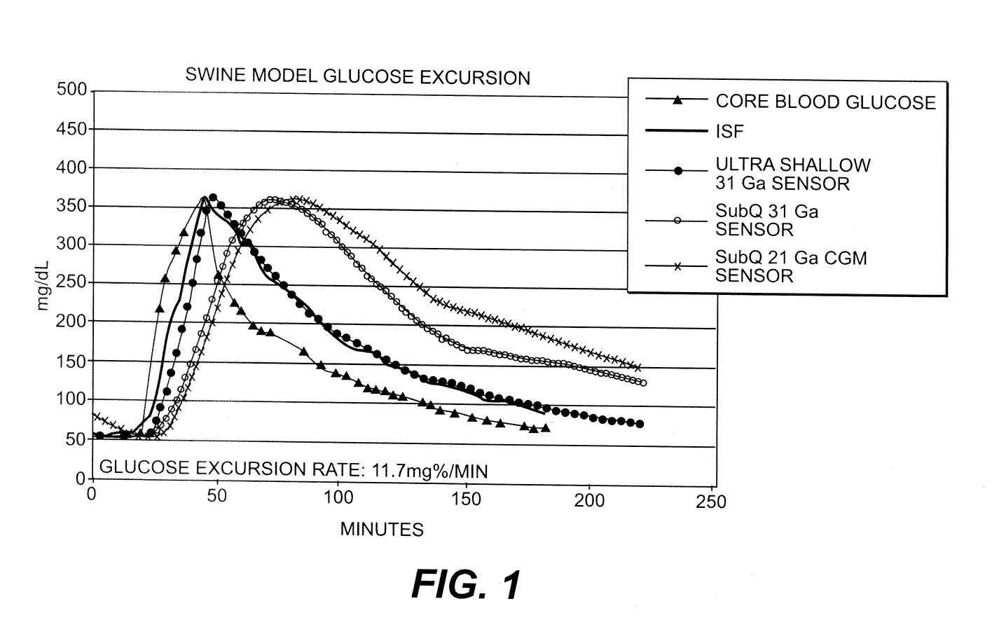

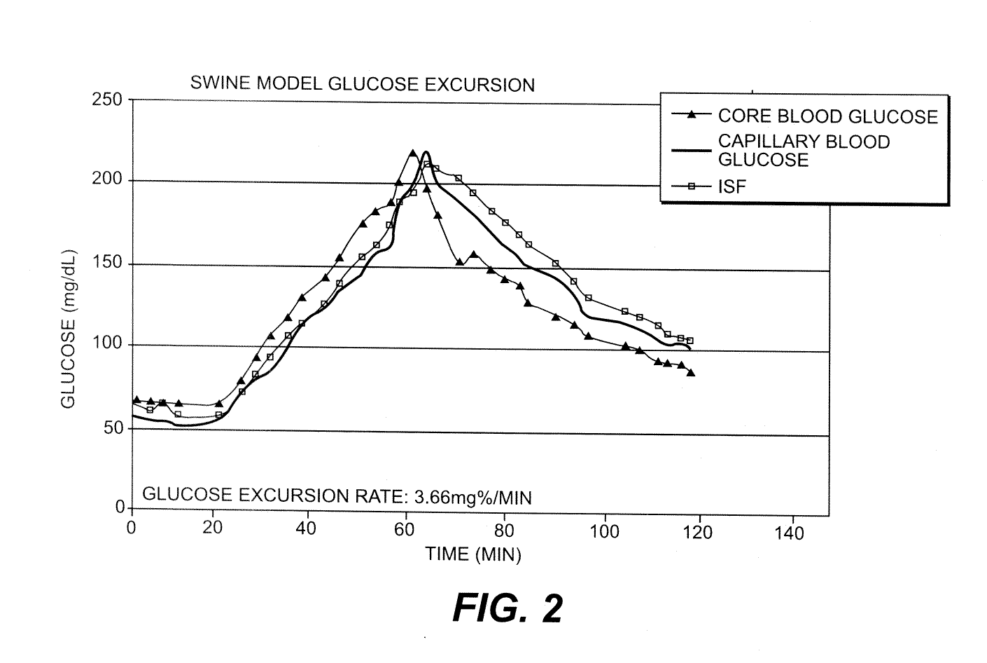

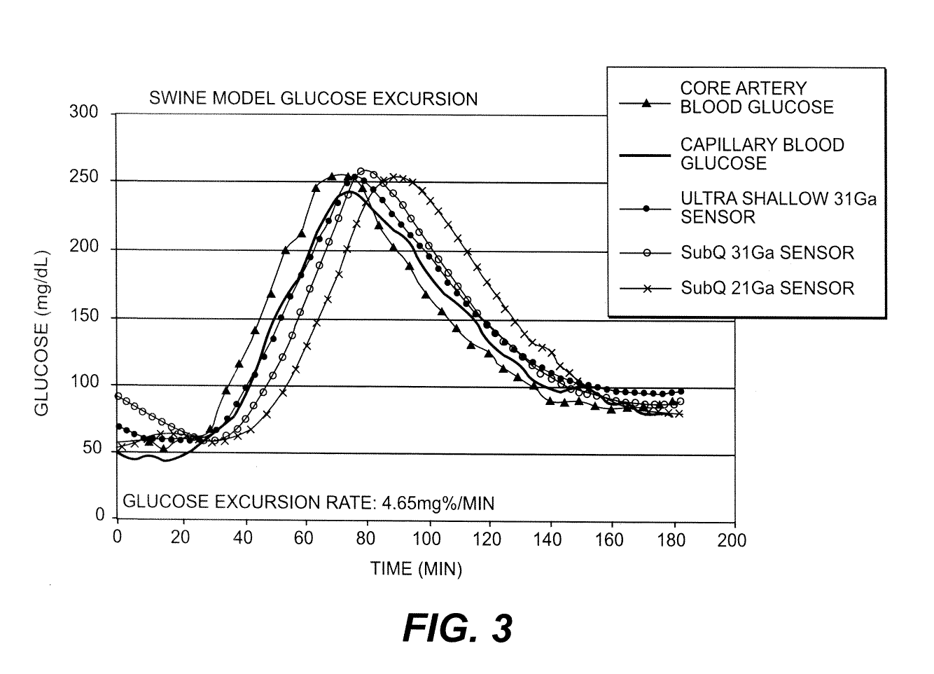

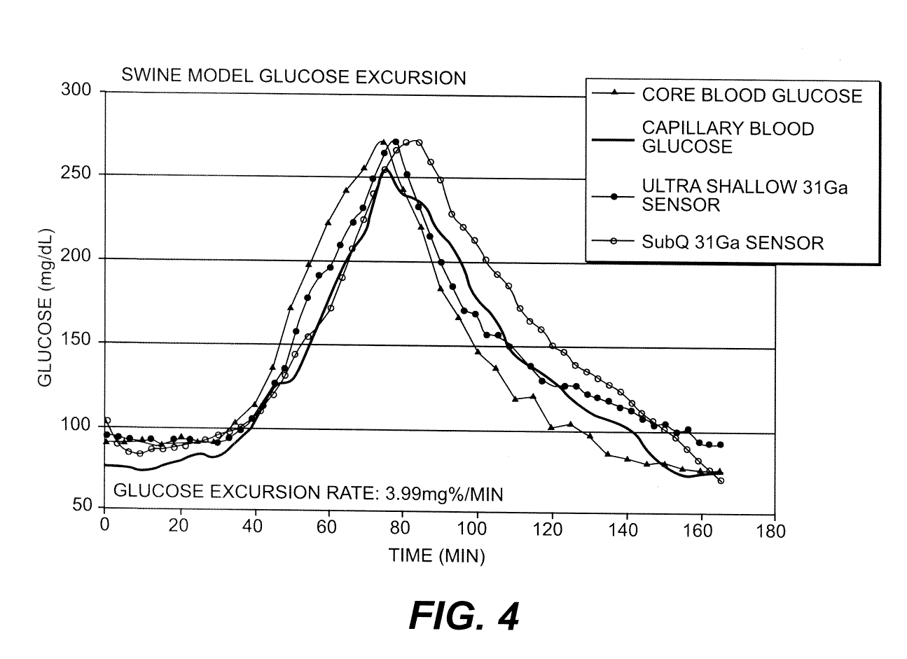

Very shallow holes were punctured (˜1 mm with a 31 Ga needle) and suction (10-15 mmHg) was used to pull ISF to the surface. The ISF fluid was then tested on an electrochemical strip that was calibrated for plasma. These ISF values were plotted against both the small sensor (FIG. 1) and capillary blood values taken from the foot (FIG. 2), and all show very good agreement. Also shown in the figures are the results of a larger sensor in the subcutaneous tissues (FIG. 3) which showed a significant time lag between the blood glucose values and the sensor readings. To establish whether the response observed was due to smaller size or shallow placement, the 31Ga sensor was placed deep in the subcutaneous tissue. The time lag observed increased with the deep SubQ placement of the 31 Ga sensor (FIG. 4). In addition, the proper depth of sensor placement was also assessed simply by observing the response in relation to the other sensors.

### Example 2

### Glucose Tracking in a Subject Under General Anesthesia

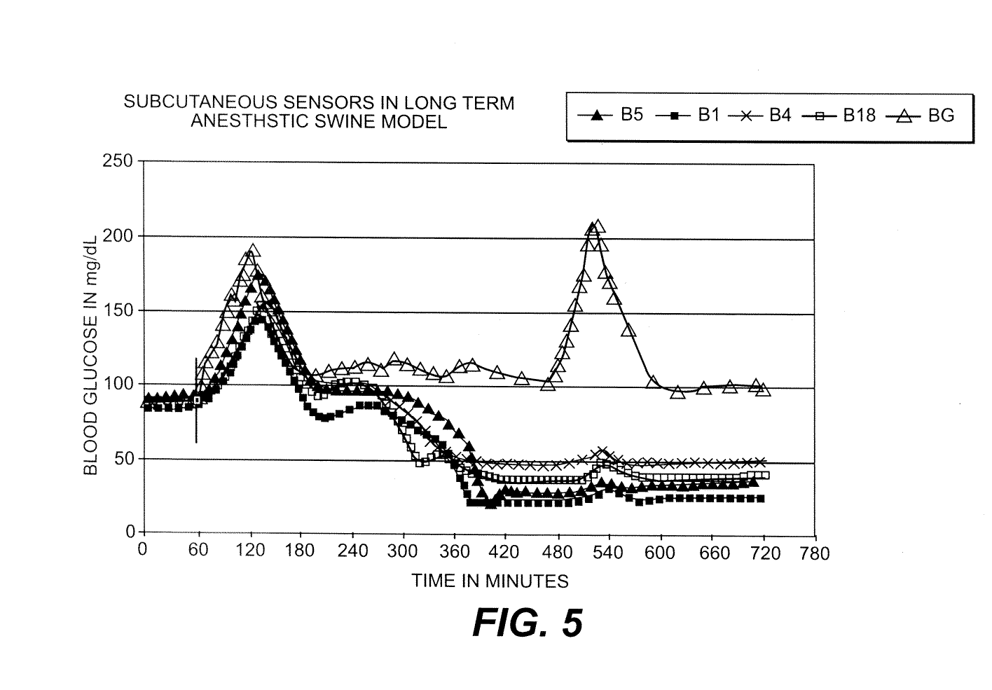

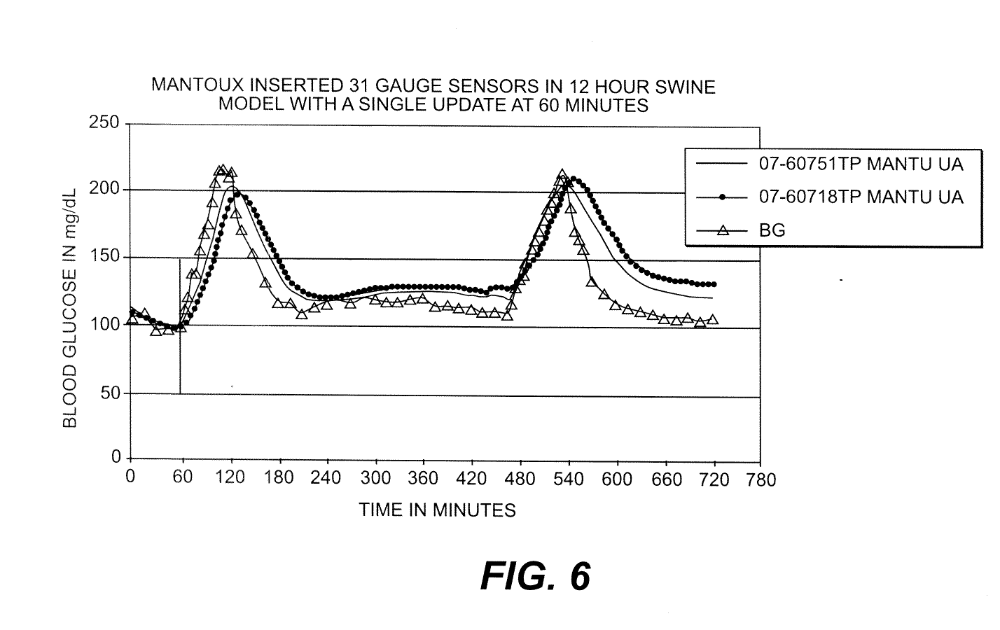

FIGS. 5 and 6 show the relative response of different sensors implanted in a pig under general anesthesia. The larger sensors, out of necessity were implanted in the subcutaneous layer of skin, tracked glucose poorly after being implanted 4 hours. The smaller sensors implanted into the intradermal space of the pigs tracked glucose well for the duration of the experiment.

What is claimed is:

1. A method for determining the blood levels of glucose in a subject, the method comprising
a) inserting in the skin of the subject to a depth of up to about 2 mm from the surface of the skin, a sensor that produces a detectable signal in the presence of glucose in the interstitial fluid of the subject,
b) maintaining said sensor in the skin at said depth such that at least a portion of said sensor is exposed to the interstitial fluid in the skin,
c) measuring the detectable signal of the sensor produced by said sensor upon exposure to said interstitial fluid, and
d) correlating the signal produced by the sensor to the level of blood glucose, thereby determining the levels of blood glucose in the subject.

2. The method of claim 1, wherein the sensor comprises a glucose-galactose binding protein (GGBP).

3. The method of claim 2, wherein the GGBP is labeled with a signaling moiety that emits an optical signal.

4. The method of claim 1, wherein a needle houses the sensor, said needle comprising a body with a proximal end and a distal end, wherein the proximal end of the needle comprises a tip, said needle being a 31 gauge needle or smaller.

5. The method of claim 1, wherein the sensor is inserted into the subject at a depth of between about 0.1 mm and 2 mm below the surface the skin.

6. The method of claim 5, wherein the sensor is inserted into the subject at a depth of between about 0.1 mm and 1 mm below the surface the skin.

7. The method of claim 6, wherein the sensor is inserted into the subject at a depth of between about 0.1 mm and 0.8 mm below the surface the skin.

8. The method of claim 1, wherein the sensor remains inserted in the subject for at least one day.

9. The method of claim 1, wherein the sensor is a continuous sensor.

10. The method of claim 1, wherein the sensor produces an optical signal in the presence of glucose for at least 4 hours.

11. The method of claim 1, wherein the subject is under a physiological stress that results in decreased peripheral blood flow.

12. A biosensor for detecting blood glucose levels in a subject, the biosensor comprising
a) a sensing mechanism that produces an optical signal in the presence of glucose said sensing mechanism comprising a fluorescently labeled glucose-galactose binding protein (GGBP) entrapped within a hydrogel matrix,
b) a shallow-depth housing that encases the sensing mechanism, wherein the shallow-depth housing comprises a hollow needle, wherein said hollow needle is a 31 gauge needle or smaller and penetrates the skin of a subject to a depth of up to about 2 mm from the surface of the skin such that said sensing mechanism is exposed to interstitial fluid, and
c) an optical conduit, wherein said optical conduit is in optical connectivity with said sensing mechanism and the needle houses at least a portion of said conduit
wherein the optical signal of the sensing mechanism generated from exposure to the interstitial fluid of the subject is correlated to blood glucose levels.

13. The biosensor of claim 12, wherein the sensing mechanism provides continuous sensing of glucose levels.

14. The biosensor of claim 13, wherein the sensor produces an optical signal in the presence of glucose for at least 4 hours.

15. The biosensor of claim 12 wherein the fluorescently labeled GGBP is covalently bound to the hydrogel matrix.

16. The biosensor of claim 15, wherein the hydrogel matrix comprises poly(ethylene glycol) dimethylacrylate (PEGDMA), 2-hydroxy-2 methyl propiophenone (HMPP) and at least one acrylate, wherein the acrylate is selected from the group consisting of methacrylic acid (MAA) and methyl methacrylate (MMA).
